(19) United States
(12) Patent Application Publication
(10) Pub. No.: US 2017/0172551 A1
(43) Pub. Date: Jun. 22, 2017

(54) Fascia Closure Tool

(71) Applicant: RAO INNOVATIONS LLC., Pacific Palisades, CA (US)

(72) Inventor: Vijendra Rao, Pacific Palisades, CA (US)

(21) Appl. No.: 15/130,325

(22) Filed: Apr. 15, 2016

Related U.S. Application Data
(60) Provisional application No. 62/269,195, filed on Dec. 18, 2015.

(51) Int. Cl. A61B 17/00; A61F 2/00; A61B 17/10

(52) U.S. Cl. CPC A61B 17/0057; A61B 17/10; A61F 2/0063; A61B 2017/00637; A61B 2017/00659; A61B 2017/00676; A61F 2002/0072

(57) ABSTRACT

A laparoscopic tool used for closing fascia implements a folding mechanism, similar to single-fold or double-fold umbrellas, in combination with anchors and a graft membrane. The graft membrane is connected across a number of graft supports. The graft supports are radially oriented around a shaft. A trigger-actuated runner slides along the shaft; movement of the trigger-actuated runner causes the graft supports to unfold or fold, thanks to a plurality of stretchers. The graft supports include a first arm, a second arm, and a third arm which are connected to each other by hinges, allowing them to fold and unfold. An anchor is positioned at the end of the third arm, the anchor being able to securely lode into fascia. A grip and a trigger at a handle end of the shaft allow the double-double fold mechanism to be engaged and the anchors and graft membrane to be secured to fascia.

The current application claims a priority to the U.S. Provisional Patent application Ser. No. 62/269,195 filed on Dec. 18, 2015.

### FIELD OF THE INVENTION

The present invention relates generally to medical devices used for laparoscopic closure.

### BACKGROUND OF THE INVENTION

Trocar site hernia is defined as an incisional hernia occurring after minimally invasive surgery at the trocar incision site. Trocar site hernia can lead to complications such as bowel herniation and obstruction. Routine closure of the fascia defect after laparoscopic surgery with existing instruments can be very tedious, complicated and time consuming, especially in obese patients. This device is a simple and effective way for a surgeon to close the fascia defect.

Laparoscopic instruments are specifically developed to be compatible with small apertures, as is necessitated by laparoscopic surgery. Laparoscopic surgery involves creating minimally sized incisions for operative procedures in order to obtain a number of benefits from smaller incisions. However, a drawback of laparoscopic surgery is the added difficulty in operating through such confined spaces; specialized tools and equipment are often required.

The present invention is one such related tool, using a compact and expandable mechanism to allow for fascia closures to be carried out. The present invention combines a graft membrane and anchors with a folding mechanism (for example single fold or double fold) to allow for internal fascial closure. Furthermore, the present invention may be utilized in conjunction with other materials such as bio-plugs.

### BRIEF DESCRIPTION OF THE DRAWINGS

FIG. 1 is an illustration showing the components of the present invention, with a graft membrane omitted for ease of disclosure.

FIG. 2 is another illustration showing the components of the present invention, with a handle and the graft membrane omitted for ease of disclosure.

FIG. 3 is an illustration showing the present invention inserted through a laparoscopic opening.

FIG. 4 is an illustration showing the present invention inserted through a laparoscopic opening and switched to an unfolded configuration, with anchors ready for attachment.

FIG. 5 is an illustration showing the present invention inserted through a laparoscopic opening in a folded configuration with graft membrane shown.

FIG. 6 is an illustration showing the graft membrane and anchors of the present invention in an unfolded and secured position, with a fascia being closed by the present invention.

FIG. 7 is a top view showing the anchors of the present invention in a radial configuration relative to the shaft.

FIG. 8 is a top view showing the anchors of the present invention in a tangential configuration relative to the shaft.

FIG. 9 is an illustration showing how the anchors of the present invention separate into distinct hooks for improved internal attachment.

FIG. 10 is an illustration showing the present invention being prepared for use in laparoscopic surgery.

### DETAIL DESCRIPTIONS OF THE INVENTION

All illustrations of the drawings are for the purpose of describing selected versions of the present invention and are not intended to limit the scope of the present invention.

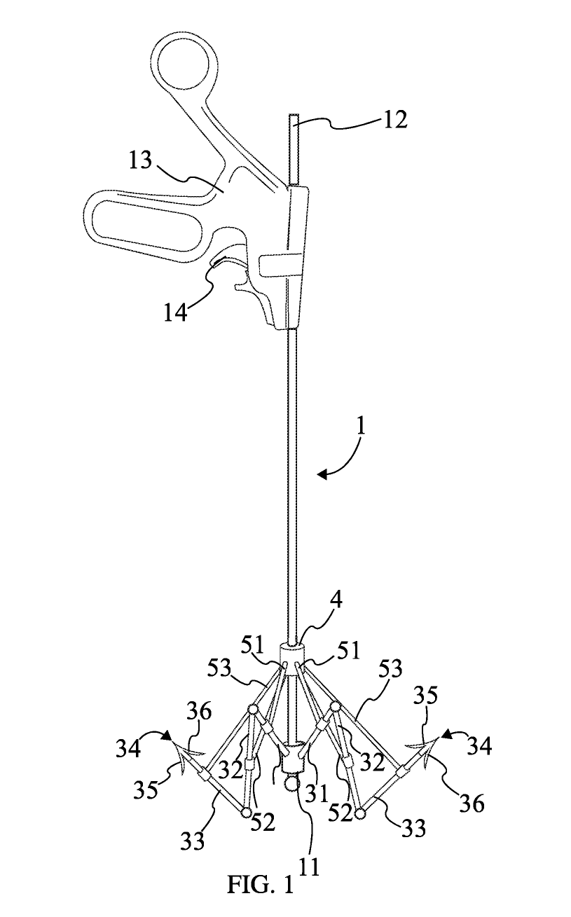

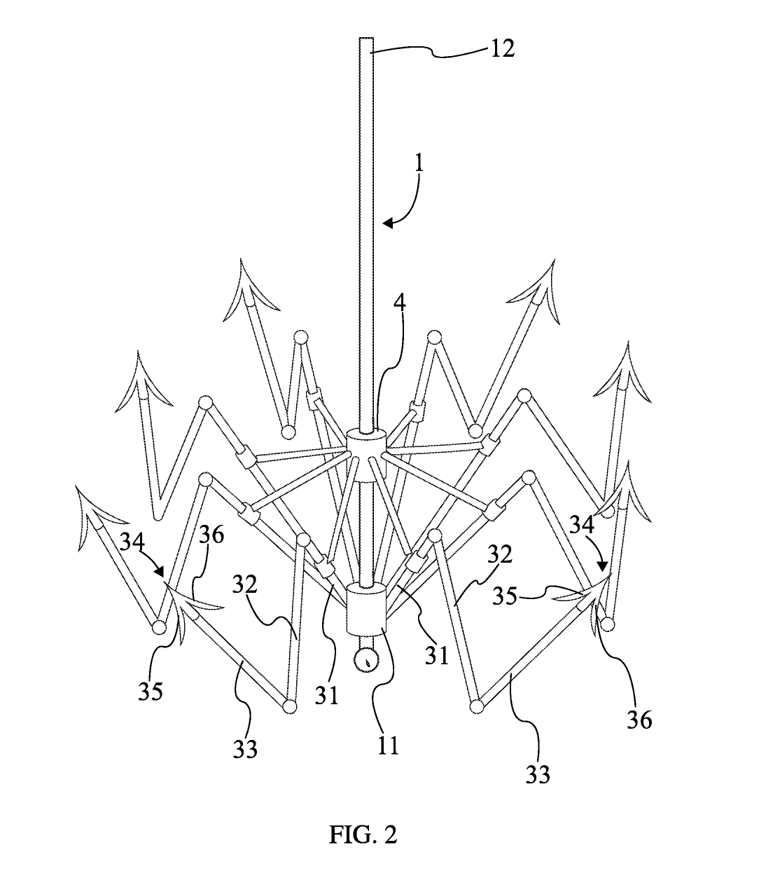

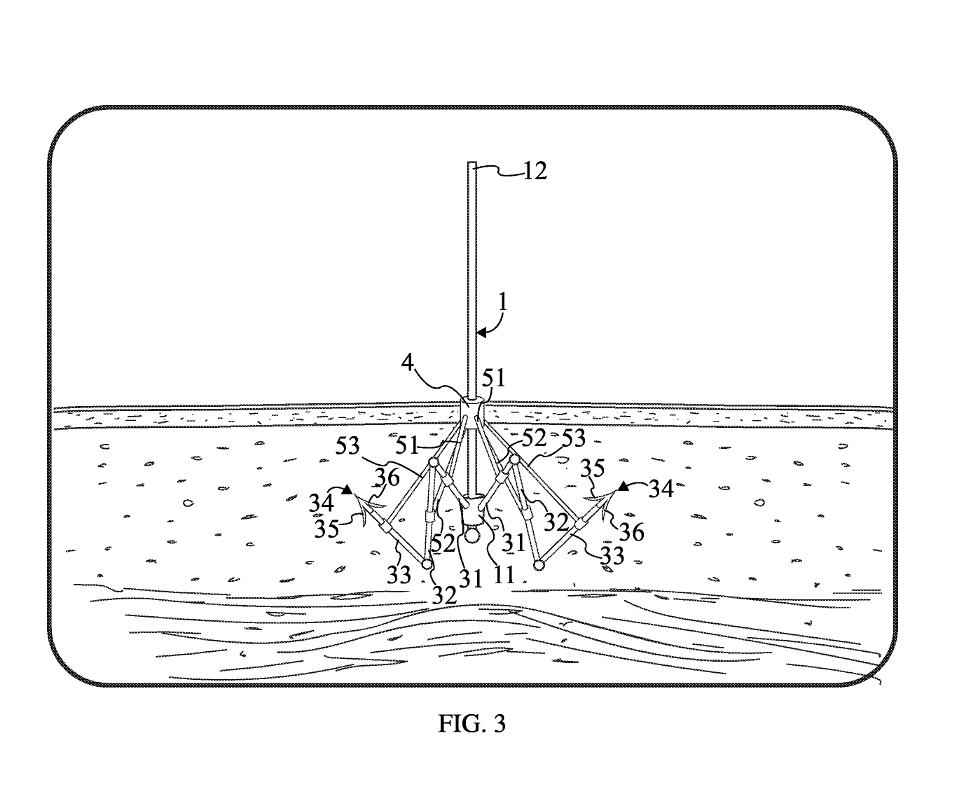

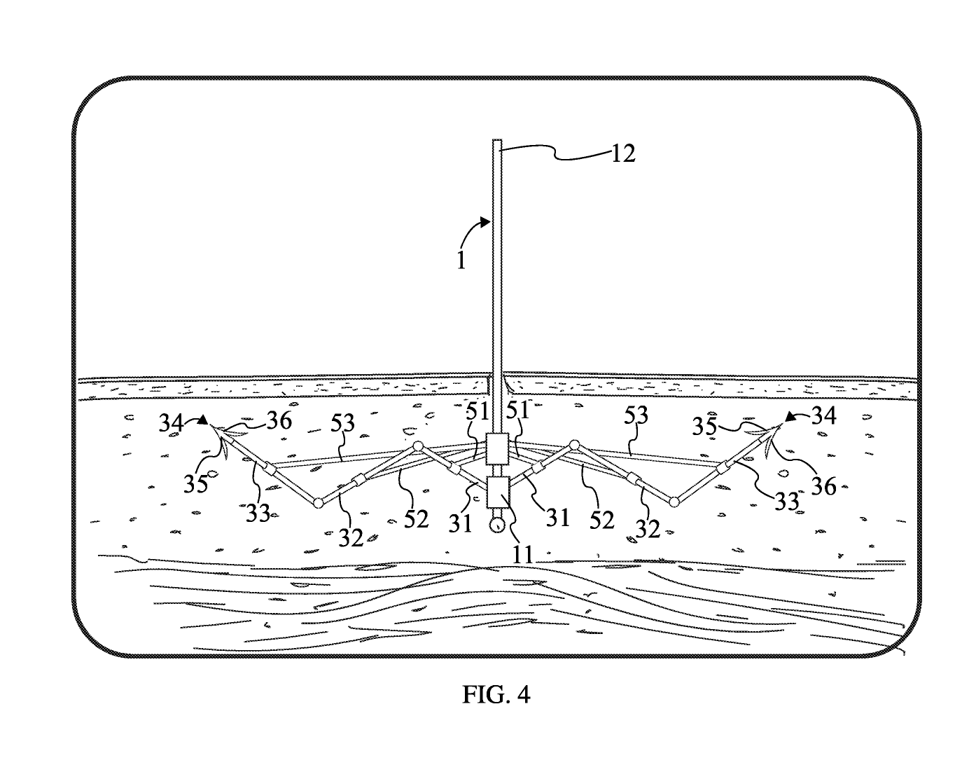

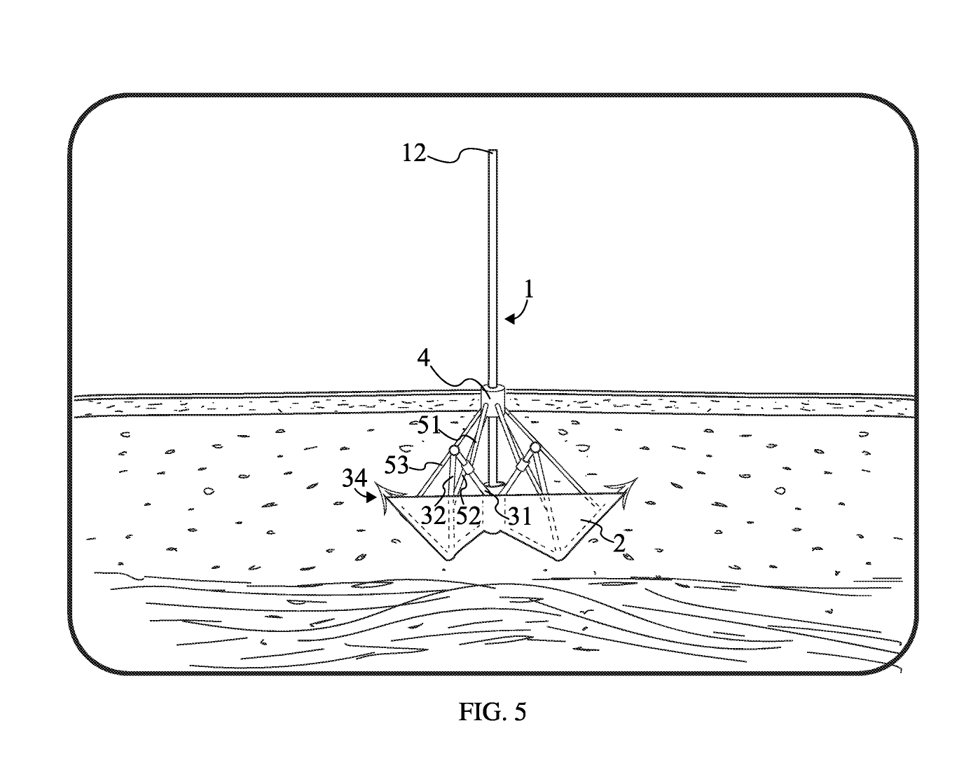

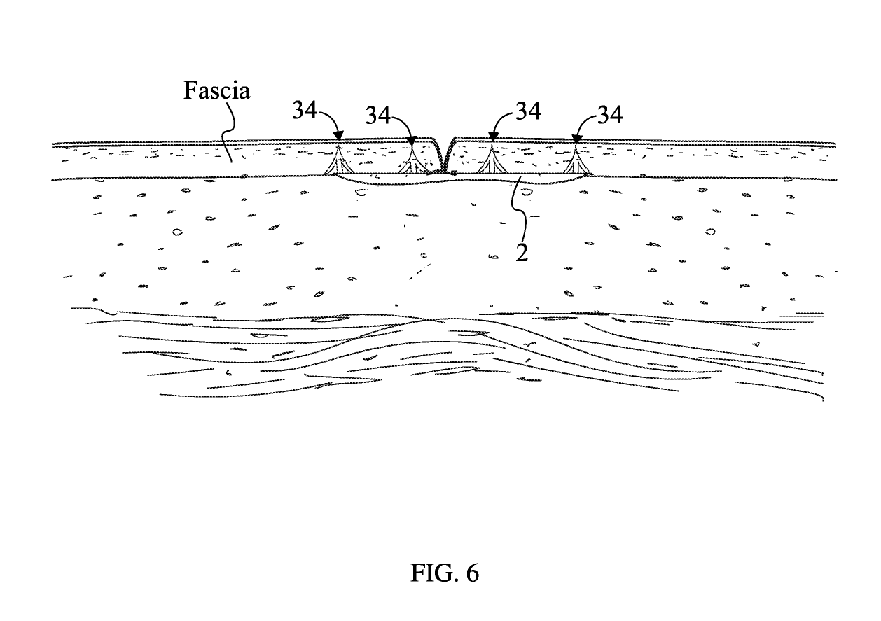

The present invention is a surgical tool that enables fascia closure, especially with respect to laparoscopic procedures. The present invention utilizes an umbrella-style folding mechanism to allow for fascia closure. To this end, the present invention comprises a shaft 1, a graft membrane 2, a plurality of graft supports 3, a trigger-actuated runner 4, and a plurality of stretchers 5. The shaft 1 allows for the present invention to be inserted through a surgical incision, while the graft membrane 2 helps close the fascia. The plurality of graft supports 3 act as ribs that receive the graft membrane 2. The plurality of graft supports 3 also provide the capability to secure the graft to the fascia or other internal anatomical structures. The trigger-actuated runner 4 connects to the plurality of graft supports 3 via the plurality of stretchers 5. Resultantly, movement of the trigger-actuated runner 4 along the shaft 1 causes the plurality of graft support to fold or unfold. The ability to fold and unfold allows the present invention to be maneuvered into tight spaces (e.g. surgical incisions) and internally fixed to a person's anatomy. The present invention is depicted in part and in full via FIG. 1-FIG. 10.

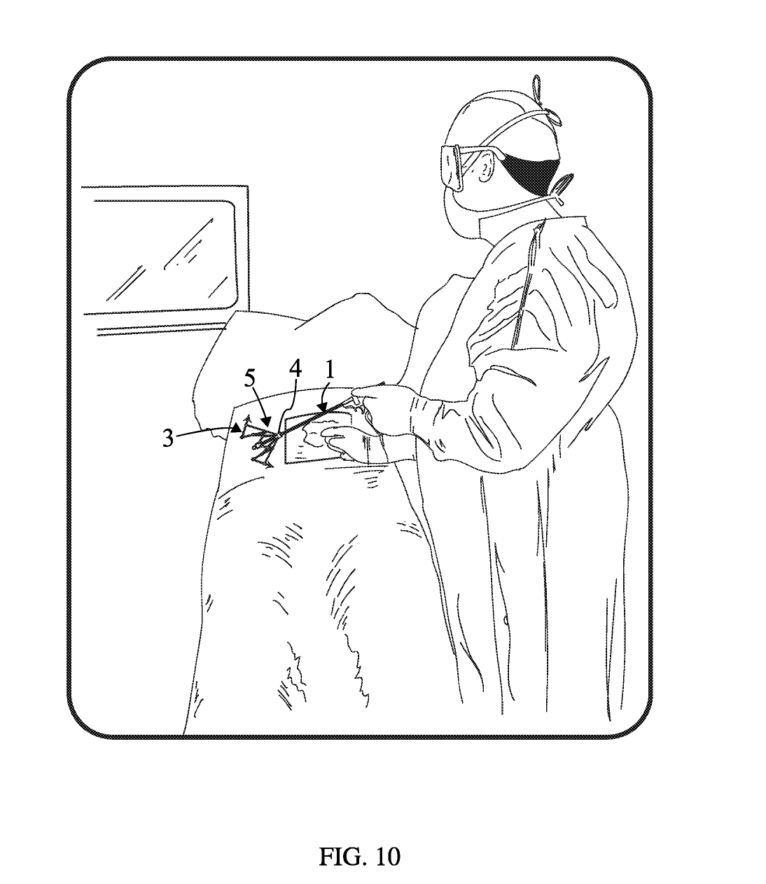

The shaft 1 comprises a stop end 11 and a handle end 12; the handle end 12 allows a person to operate the present invention while the stop constrains movement of the trigger-actuated runner 4 and resultantly the unfolding of the plurality of graft supports 3. The plurality of graft supports 3 is radially positioned around the shaft 1. The graft membrane 2, which binds and closes the fascia, is connected across the plurality of graft supports 3. The connection of the graft membrane 2 across the plurality of graft supports 3 is similar to how an umbrella's canopy is connected to said an umbrella's ribs. The trigger-actuated runner 4 is slidably engaged with the shaft 1, such that the trigger-actuated runner 4 can move between the stop end 11 and the handle end 12. Movement of the trigger-actuated runner 4 causes the present invention to fold or unfold, courtesy of the plurality of stretchers 5 connecting the plurality of graft supports 3 to the trigger-actuated runner 4. The shaft 1 shown in FIG. 1-FIG. 5 as well as FIG. 10.

While the present invention potentially can make use of single-fold and double-fold mechanisms, the preferred and illustrated embodiment implement a double-fold mechanism. A double-fold mechanism is advantageous compared to a single-fold mechanism as it creates positive tension in 3 dimensions when used for fascia closure. Each graft support of the preferred embodiment thus comprises a first arm 31, a second arm 32, a third arm 33, and an anchor 34. In support of the double-fold mechanism, the hinged connections are used to connect the arms to each other and to the stop end 11. The fold mechanism is easily seen in FIG. 1-FIG. 5.

More specifically, the first arm 31 is hingedly connected to the stop end 11 of the shaft 1. The second arm 32 is hingedly connected to the first arm 31, opposite the stop end 11. Lastly, the third arm 33 is hingedly connected to the second arm 32, opposite the first arm 31. This configuration allows for the first arm 31, second arm 32, and third arm 33 to be folded adjacently to each other and the shaft 1; this configuration is similar to a folded umbrella and its compact profile is ideal for insertion through a surgical incision. The hinged connections also allow for the present invention to be switched to an unfolded state, in which the first arm 31, second arm 32, and third arm 33 are angled away from the shaft 1 and thus suited for mounting to an anatomical feature by means of the anchor 34.

The anchor 34 lodges into an anatomical feature (e.g. fascia) in order to hold the graft support and connected graft membrane 2 in place. In order to make sure the anchor 34 is not obstructed by other components of the present invention, the anchor 34 is adjacently connected to the third arm 33, opposite the second arm 32. This positioning, in combination with the unfolding capability of the graft support, allows for the double-fold mechanism to be engaged in order to lodge the anchor 34 in the fascia.

Returning to the graft supports 3, it is necessary for a connecting member to impart folding of the arms as a result of movement of the trigger-actuated runner 4. To this effect, a plurality of stretchers 5 are hingedly connected between the trigger 14 actuated runner 4 and the plurality of graft supports 3. These three components effectively form a triangle; thus by changing the angle between the shaft 1 and the plurality of stretchers 5, the angle between the shaft 1 and the plurality of graft supports 3 is also changed. Changes in angle are created by movement of the trigger-actuated runner 4 as subsequently elaborated upon.

The plurality of stretchers 5 comprises a primary stretcher 51 in order to join engage the trigger-actuated runner 4 with the first arm 31. The primary stretcher 51 is hingedly connected to both the trigger-actuated runner 4 and the first arm 31. Resultantly, a triangular shape is created between the shaft 1, the primary stretcher 51, and the first arm 31. While said triangular shape is adjustable thanks to the trigger-actuated runner 4, some constraints remain in place. Most notably, the sum of the angles of the triangle always adds to 180 degrees. Thus, as the trigger-actuated runner 4 moves closer to the stop end 11, the angle between the shaft 1 and the primary stretcher 51 increases. To compensate for this, the angle between the stretcher and the first arm 31 must decrease. As a result, the primary stretcher 51 causes the first arm 31 to extend outwards when the trigger-actuated runner 4 is moved towards the stop end 11 of the shaft 1.

Inversely, when the trigger-actuated runner 4 moves away from the stop end 11 of the shaft 1, the angle between the shaft 1 and the primary stretcher 51 decreases. To compensate for this, the angle between the first arm 31 and the primary stretcher 51 must increase, resulting in the first arm 31 being moved towards the shaft 1 into a “folded” configuration.

By providing a hinged connection between the trigger-actuated runner 4 and the first arm 31 (via the primary stretcher 51), a single-fold mechanism is created. While, potentially, the anchors 34 could be connected to the first arm 31 in order to make use of a single fold mechanism, a double-fold mechanism is more advantageous as earlier discussed.

As a double-fold mechanism is preferable for the present invention, the plurality of stretchers 5 further comprises a secondary stretcher 52 and a tertiary stretcher 53. The secondary stretcher 52 is hingedly connected between the trigger-actuated runner 4 and the second arm 32. As a result, movement of the trigger-actuated runner 4 imparts movement to the second arm 32. As the second arm 32 is also hingedly connected to the first arm 31, the trigger-actuated runner 4 causes the second arm 32 to rotate either towards or away from the first arm 31.

The third arm 33, likewise, can be rotated thanks to the tertiary stretcher 53 being hingedly connected between the trigger-actuated runner 4 and the third arm 33. As the trigger-actuated runner 4 is moved towards the stop end 11 of the shaft 1, the tertiary stretcher 53 pushes the third arm 33 and rotates said third arm 33 away from the second arm 32, into an “unfolded” configuration.

Effectively, the interaction of the primary stretcher 51 between the trigger-actuated runner 4 and the first arm 31 is replicated for the second arm 32 and the third arm 33. The trigger-actuated runner 4 moves the second arm 32 via the secondary stretcher 52, and likewise moves the third arm 33 via the tertiary stretcher 53. Resultantly, switching the present invention between a folded configuration and an unfolded configuration is easily accomplished by a medical professional or operating entity in general. The folded configuration allows for easy insertion into a surgical incision while the unfolded configuration allows for the anchors 34 and graft membrane 2 to be internally secured in order to close a fascia.

Describing the anchor 34 in more detail, the anchor 34 comprises a left hook 35 and a right hook 36. These hooks are provided to allow the anchor 34 to easily be inserted into the fascia, as well as to secure the anchor 34 in position once inserted into the fascia. The left hook 35 and the right hook 36 each comprise a tip 37 and a barb 38. The tip 37 pierces the fascia to allow the anchor 34 to be inserted, while the barb 38 lodges within the fascia. The barb 38 holds the anchor 34 within the fascia, preventing the anchor 34 (and thus the graft membrane 2) from becoming dislodged. The tip 37 is the portion of the hook that is adjacently connected to the third arm 33, while the barb 38 is adjacently connected to the tip 37. The barb 38 is at an acute angle 39 relative to the third arm 33.

The preferred embodiment of the present invention makes use of two hooks in order to improve deployment of the graft supports 3 and the graft membrane 2. As the left hook 35 and the right hook 36 are connected to the third arm 33, but not connected to each other, sufficient tension in the graft membrane 2 and the third arm 33 (for example as created when switching the present invention to an unfolded configuration) causes the left hook 35 and the right hook 36 to separate from each other. This improves lodging of the anchor 34 within the fascia.

The orientation of the left hook 35 and the right hook 36 can be changed for different embodiments of the present invention. For example, in one embodiment of the present invention, the left hook 35 and the right hook 36 are oriented to be coincident with the shaft 1. That is, the left hook 35 and the right hook 36 both fall on a radial line extended from the center of the shaft 1.

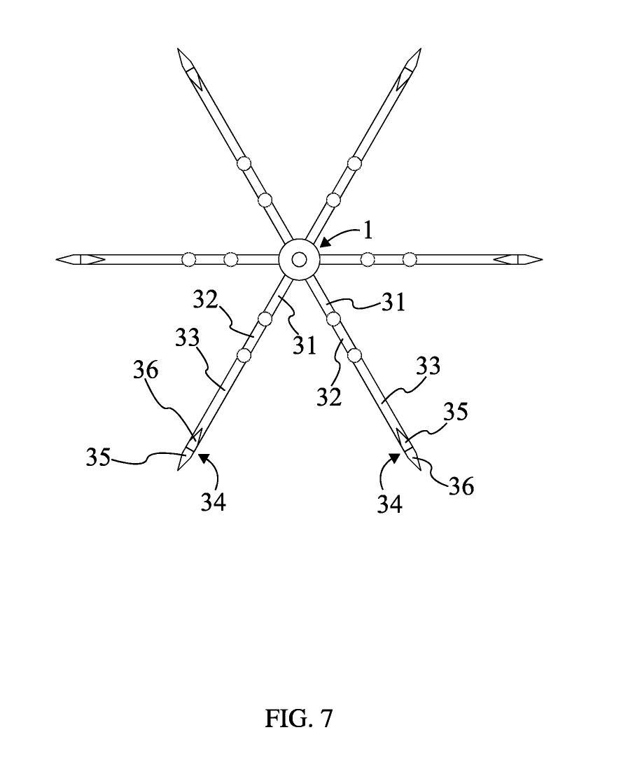

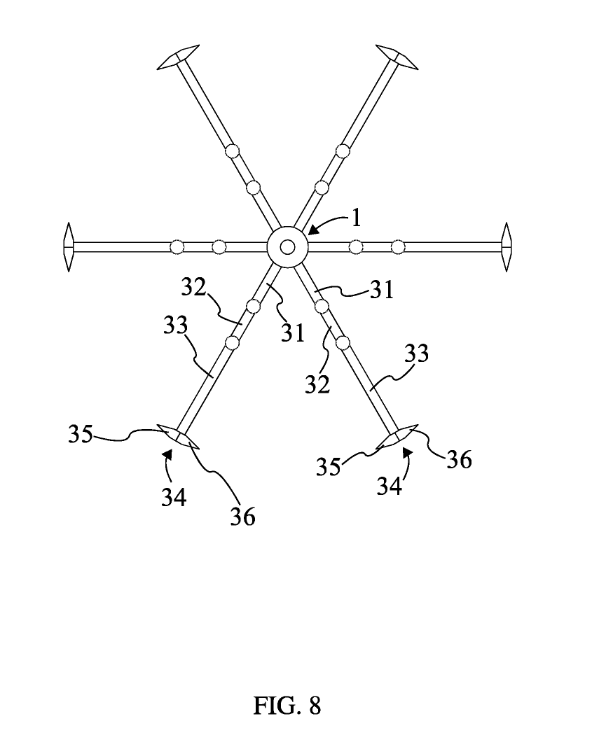

Alternatively, in another embodiment, the left hook 35 and the right hook 36 are instead oriented perpendicular to the shaft 1. In other words, the left hook 35 and the right hook 36 are positioned along a line that is parallel to a tangent line of the shaft 1. These two configurations are shown via FIG. 7 and FIG. 8, respectively.

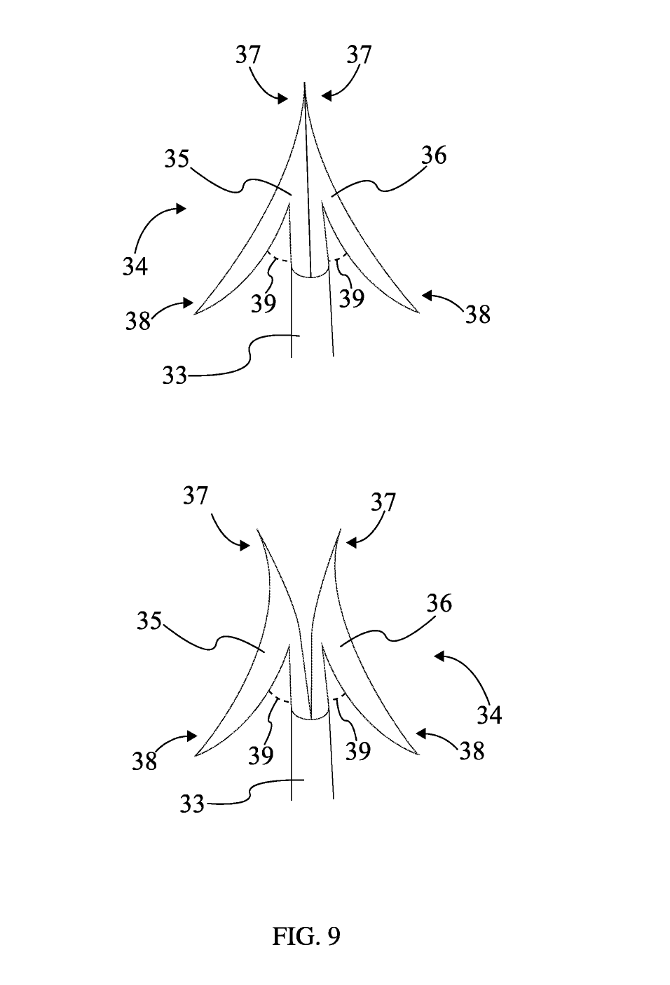

Further orientations of the left hook 35 and the right hook 36 relative to the shaft 1 are possible, though the two examples described above are more common. Regardless of the orientation of the left hook 35 and the right hook 36, both the left hook 35 and the right hook 36 are each connected to the graft membrane 2. This allows the graft membrane 2 to separate the left hook 35 and the right hook 36 via tension. The tension, as earlier described, is created when the present invention is switched to an unfolded configuration, with the first arm 31, second arm 32, and third arm 33 being extended away from the shaft 1. Separation of the anchors 34 is shown in FIG. 9.

As the present invention is intended to be positioned through a surgical incision, it is preferable to provide a means of switching the present invention between a folded and an unfolded configuration in order to allow the present invention to be operated outside of the incision. To this end, the shaft 1 further comprises a grip 13 and a trigger 14. The grip 13 is positioned at the handle end 12 of the shaft 1, while the trigger 14 is mounted into the grip 13. The grip 13 not only provided a an ergonomic interface for the present invention, it also allows a user to easily maneuver and insert the present invention into a surgical incision, all of this being accomplished external from the surgical incision. Use of the present invention in a surgical environment is depicted via FIG. 10.

The trigger 14, which controls movement of the trigger-actuated runner 4, is mounted onto the grip 13 in order to be easily accessed by a user without having to move their hand from the grip 13. A user can thus grasp the handle, insert the shaft 1 through a surgical incision, then engage the trigger 14 to activate the double-fold mechanism in order to switch the present invention to an unfolded configuration. The trigger 14 itself is operatively coupled to the trigger-actuated runner 4, such that a surgeon can use the trigger 14 to control movement of the trigger-actuated runner 4. A surgeon is thus capable of externally closing a fascia, via anchors 34 and a graft membrane 2, thanks to the present invention.

The specific means by which the trigger 14 controls movement of the trigger 14 actuated-runner 4 is not restricted by the present invention. For example, one possible mechanism is connecting a spring to the trigger-actuated runner 4. This spring will cause the trigger-actuated runner 4 to return to or maintain a default (i.e. unfolded configuration) unless acted upon by an external force. The trigger 14, meanwhile, may simply be connected to the trigger-actuated runner 4 by a rod, such that pressing the trigger 14 moves the rod down, and resultantly moves the trigger-actuated runner 4 towards the stop end 11 of the shaft 1. When the trigger 14 is released, the spring causes the trigger-actuated runner 4 to return to its default position.

As another example, the trigger 14 can rotate a cam which is connected to the trigger-actuated by a rod. Rotating the cam moves the rod either up or down, with said rotation either pushing the trigger-actuated runner 4 down (for an unfolded configuration) or pulling the trigger-actuated runner 4 up (for a folded configuration). Further means of coupling the trigger 14 with the trigger-actuated runner 4 remain possible within the scope of the present invention.

The present invention is not restricted to the above described embodiments; additions, subtractions, and substations remain possible within the scope of the present invention. As one example, a cautery mechanism can be integrated into the anchor 34. The cautery mechanism eases the process of lodging the anchor 34 within the fascia. The cautery mechanism could be a caustic substance, a high temperature substance, or other integrated tool or coating that facilitates cauterization of the fascia.

A further possibility is the use of different shapes for the anchors 34. For example, flat anchors 34 can be used to allow for an application of a biodegradable glue. This biodegradable glue then allows for the anchors 34 to be bonded to the fascia, securing the graft membrane 2 to the fascia. Potentially, a biodegradable glue could also be combined with a pointed anchor 34, though the surface provided by a pointed anchor 34 is less ideal for receiving a biodegradable glue.

Although the invention has been explained in relation to its preferred embodiment, it is to be understood that many other possible modifications and variations can be made without departing from the spirit and scope of the invention as hereinafter claimed.

What is claimed is:

1. A fascia closure tool comprises:
a shaft;
a graft membrane;
a plurality of graft supports;
a trigger-actuated runner;
a plurality of stretchers;
the shaft comprises a stop end and a handle end;
the plurality of graft supports being radially positioned around the shaft;
each of the plurality of graft supports comprises a first arm, a second arm, a third arm, an anchor;
the graft membrane being connected across the plurality of graft supports;
the trigger-actuated runner being slidably engaged with the shaft between the stop end and the handle end;
the first arm being hingedly connected to the stop end;
the second arm being hingedly connected to the first arm, opposite the stop end;
the third arm being hingedly connected to the second arm, opposite the first arm;
the anchor being adjacently connected to the third arm, opposite the first arm; and
the plurality of stretchers being hingedly connected between the trigger-actuated runner and the plurality of graft supports.

2. The fascia closure tool as claimed in claim 1 comprises:
the plurality of stretchers comprises a primary stretcher; and
the primary stretcher being hingedly connected between the trigger-actuated runner and the first arm.

3. The fascia closure tool as claimed in claim 1 comprises:
the plurality of stretchers comprises a secondary stretcher; and
the primary stretcher being hingedly connected between the trigger-actuated runner and the second arm.

4. The fascia closure tool as claimed in claim 1 comprises:
the plurality of stretchers comprises a tertiary stretcher; and
the primary stretcher being hingedly connected between the trigger-actuated runner and the third arm.

5. The fascia closure tool as claimed in claim 1 comprises:
the anchor comprises a left hook and a right hook;
the left hook and the right hook each comprise tip and a barb;
the tip being adjacently connected to the third arm; and
the barb being adjacently connected to the tip.

6. The fascia closure tool as claimed in claim 5 comprises:
the left hook and the right hook being oriented coincident to the shaft.

7. The fascia closure tool as claimed in claim 5 comprises:
the left hook and the right hook being oriented perpendicular to the shaft.

8. The fascia closure tool as claimed in claim 5 comprises:
the barb being oriented at an acute angle from the third arm.

9. The fascia closure tool as claimed in claim 5 comprises:
the left hook being connected to the graft membrane; and
the right hook being connected to the graft membrane.

10. The fascia closure tool as claimed in claim 1 comprises:
the shaft further comprises a grip and a trigger;
the grip being positioned at the handle end; and
the trigger being mounted into the grip.

11. The fascia closure tool as claimed in claim 11, wherein the trigger is operatively coupled to the trigger-actuated runner.
